(19) United States
(12) Patent Application Publication
(10) Pub. No.: US 2009/0074847 A1
(43) Pub. Date: Mar. 19, 2009

(54) Transport System In Biological Systems

(75) Inventors: Jurgen Wilhelm Richard Bernhardt, Weinstadt (DE); Felix Oliver Heinrich, Esslingen (DE); Karin Engelhart-Jentzsch, Ludwigshafen (DE)

Correspondence Address: COLLARD & ROE, P.C., 1077 NORTHERN BOULEVARD, ROSLYN, NY, 11576, US

(73) Assignee: BIO TESYS GMBH, Esslingen (DE)

(21) Appl. No.: 10/512,737

(22) PCT Filed: Apr. 16, 2003
(86) PCT No.: PCT/EP03/03973

§ 371 Date: Jun. 16, 2005

(30) Foreign Application Priority Data
Apr. 29, 2002 (AT) A 656/2002

(51) Int. Cl. A61K 9/127; A61K 35/32; A61K 33/42; A61K 38/43

(52) U.S. Cl. 424/450; 424/548; 424/601; 424/94.1; 426/648

(57) ABSTRACT

The invention relates to a transport system (1) for active substances containing hybrid particles (2) comprising at least one layer of lipid molecules (3) and at least one ligand, the ligand being a peptide (5).

The invention relates to a transport system for substances containing hybrid particles, comprising at least on layer of lipid molecules and at least one ligand, which is a peptide.

The invention further relates to a method of transporting substances.

The invention additionally also relates to a transport system for use as a medicament.

The invention further relates to the use of the transport system to produce a medicament for the treatment of nutritional deficiencies.

These days, a plurality of illnesses, such as chronic or acute inflammatory processes, infections, ischaemia, etc., are known to be caused by general deficiencies or pathophysiological states, which are characterised by a systemic or local deficiency of micro-nutrients. The transport of micro-nutrients is a major requirement for normal cellular function and hence for maintaining and regenerating cell bonds. Of the micro-nutrients, liposoluble vitamins are also of central importance to the perfect functioning of general physiological processes. Their metabolism is subject to very efficient control and often also to homeostatic regulation. This physiological balancing conflicts in a certain way in the case of indications for which it would be advisable from a medicinal and in particular nutritional medicinal point of view to apply local high concentrations of micro-nutrients. In the case of such indications, the problem of the systemic balance can only be addressed by administering very high concentrations of micro-nutrients. However, this can lead to too high a strain on the organism as a whole to some extent, in particular on the liver but also the kidneys, through which the high concentration of micro-nutrients circulates.

One transport option is that of hydrophilic polymer gels of natural and synthetic origin (so-called hydrogels, e.g. alginate, fibrin, poly-lactate-glycolic acid copolymer (GLGLA) etc.). However, it is not easy to transport hydrophobic substances using these polymer materials because polymer hydrogels are generally substances which swell in contact with water and it is not possible to charge them with hydrophobic micro-nutrients (such as liposoluble vitamins or vitamin precursors, for example), without undertaking further modifications.

Liposomes, which carry short polymer chains at their surface which swell in water (such as polyethylene glycol (PEG)) also remain stable over a long period. This can be attributed to the entropy elasticity of whatever polymer is used at the surface of the liposomes. The first molecules which make initial contact with the particles used in a therapeutic treatment are proteins. A signal cascade is triggered due to adsorption of these proteins, which is what ultimately causes the targeted foreign bodies to be “eliminated” by the immune system. The adsorption of proteins on the surface of liposomes, which carry short polymer chains, causes these polymers to be compressed. By restricting the conformation freedom in this situation, the polymer consumes a very large amount of energy in the form of entropy. The polymer deals with this state, which is detrimental in terms of energy, by extending again, which causes the protein to be repelled, in the manner of a spring which is compressed and then relaxed again. This process therefore prevents proteins from being adsorbed and ultimately causing the immune system to react. Parameters which influence this repulsion process are the surface concentration and the length (molecular weight) of the polymers as well as the phase behaviour in mixtures with other molecular elements of the liposomes.

Human studies have shown that liposome systems in which PEG-modified lipids are incorporated have a half-life of up to 45 hours. Many administered substances in which liposomes and polymer-functionalised liposomes are used are based on a passive introduction of the substances into specific tissue. This means that a therapeutic improvement ultimately depends on the particles remaining in the body for long periods. On this premise, however, there is no way of ruling out a situation in which the substances will ultimately concentrate at the same time in various different tissues, including those where the substance is not required or in the worst case scenario could also cause undesired side-effects.

Liposome structures constitute a suitable carrier system for substances and enable such substances to be transported and released. Precisely transport systems made with a base of liposomal structures have become increasingly common in medical treatments in recent years and are already used widely on a commercial basis. Use is made of the fact that small uni-lamellar vesicles (diameter of less than 100 nm) are not sufficiently detected by the human immune system and can not therefore be destroyed by macrophages or monocytes. Consequently, the dwell time (half-life times) of these formulations is significantly increased, which is an enormous advantage from a therapeutic point of view. In addition to structure-geometric factors which can lead to increased stability over long periods, this same effect can also be achieved by appropriate adjustment and control of the physical-chemical surface properties (such as charge, for example) of the particulate systems. In one example, the substance daunorubicin citrate is packed in passive, i.e. “non”-functionalised liposomes and used to treat Kaposi's sarcoma lesions. (Nunez, M., Saballs, P., Valencia, M E., Santos, J., Ferrer, E., Santos, I., Berrocal, A., Galindo, M J., Podzamczer, D., Gonzalez-Lahoz, J. 2001. Response to liposomal doxorubicin and clinical outcome of HIV-1-infected patients with Kaposi's sarcoma receiving highly active antiretroviral therapy. HIV Clin. Trials. 2(5):429-37.) The disadvantage of this approach is that the targets can not be directly supplied with substances without placing strain on the organism as a whole. This is only made possible by bypassing regulation mechanisms.

Accordingly, the objective of the invention is to propose a way of selectively transporting substances in a biological system.

The objective of the invention is achieved by the transport system having the features defined in the characterising part of claim 1. The advantage of the transport system resides in the fact that by combining the lipid molecule with a peptide, the system not only remains in the body for an appropriate length of time, it is also actively able to communicate with cells. Another advantage is the fact that transport systems which contain a defined composition and interaction of molecular modules as well as controllable structures in the submicrometre range, can absorb additional micro-nutrients in specific compartments and transport them selectively into specific tissue types. Such particulate transport systems which have a structural/functional definition and are simultaneously able to communicate actively with biological tissue in a controllable manner, represent one of the major opportunities of biotechnology. Another advantage is the fact that the substances can be accumulated at the site where action is needed in order to produce a high concentration at this site rather than causing systemic strain on the organism as a whole.

Another embodiment defined in claim 2 is of advantage, because steric obstacles and/or charge interference in the interaction of the hybrid particle with the bonding partner, in particular a target cell, can be prevented by inserting a spacer unit between the polar head group of the lipid molecule and the peptide.

As defined in claim 3, the spaced layout of the oligopeptides enables the particulate system to bone with both bigger and non-adjacent bonding partners because there are no obstacles caused by a lack of space preventing the possibility of extension.

It has proved to be of advantage, as defined in claim 4, if molecules can be used which are not detected by the body's immune system or are so to only a certain extent, so that they will not be destroyed. It is also of advantage if these molecules possess the property of being able to organise themselves into vesicular structures of their own accord.

Other embodiments defined in claims 5 and 6 are of advantage whereby interactions are induced with peptides forming parts of the hybrid particles of the transport system and which are disposed externally to the cell, by means of receptors which are anchored in the cell as trans-membrane proteins. Incorporating such peptides in synthetic bio-materials of a defined structure and modifying the boundary surfaces of particulate synthetic transport systems offers the possibility of being able to influence the interactions of these bio-mimetic materials with the target cells.

In the embodiment defined in claim 7, the active substances can be selectively transported to the site where they are intended to act and do not therefore place strain on the entire organism. This also prevents undesired side-effects caused by active substances reaching all cells, which is to be feared if the worst case scenario were to occur.

This being the case, the embodiment defined in claim 8 is of advantage, whereby these sequences enable a targeted delivery of active substances to the cells of the eye, in particular the cells of the retina. It has also been found that the targeting accuracy of the transport system can be improved by combining several oligopeptide sequences, thereby resulting in another advantage.

The embodiment of the transport systems defined in claim 9 has proved to be of advantage due to the fact that the active substances can b e transported directly to the target cells, in particular the skin cells, preferably to fibroblasts, thereby preventing any strain on other cells and hence other organs. In this respect, a combination of several different sequences has proved to be of particular advantage because the active substances can be transported even more accurately.

This being the case, another advantage defined in claim 10 is the fact that the hybrid particles do not have to be disposed along a substrate but automatically assume 3-dimensional structures. The layout of the hybrid particles has also been shown to be of advantage because active substances can be packaged and thus protected from metabolic processes on the way to the intended site of action. Another advantage is the fact that the active substances are packaged in a particulate transport system, which prevents immunological attacks and degradation processes, thus ensuring that the active substances reach the site of intended action in their original form.

As defined in claim 11, the release of the micro-nutrients can be controlled by the cross-linking of the hybrid particles by means of polymerisable groups. Apart from the ability to remain in a specific tissue for a long period of time, the fact that the physical-chemical surface properties, in particular the concentration and presentation of a peptide at the boundary surface, can be controlled represents another important criterion for the successful use of active particulate transport systems for active substances.

Also of advantage is the embodiment of the transport system defined in claim 12, which enables many different substances to be transported. The transport of one of these substances or a combination of these substance is an important aspect in the prophylactic treatment of illnesses and regeneration. By supplying these substances, the progress of illnesses can be positively controlled, especially in the case of chronic illnesses. Another advantage is the fact that a much broader therapeutic range can be obtained by delivering several micro-nutrients often, because synergetic effects often result from combinations of several micro-nutrients, which is what to a certain extent makes the pharmacology of micro-nutrients significantly different from other drugs. For example, it can be shown that no oxidative protection is afforded by supplying only one micro-nutrient, for example, whereas a combination of two or more micro-nutrients with anti-oxidative properties can be shown to have an antioxidant effect.

The embodiment defined in claim 13 is also of advantage because providing micro-nutrients assists in the prevention of illnesses and helps to regenerate the organism. The orthomolecular properties of the micro-nutrients triggers biochemical irritants, which can be meaningfully evaluated by the organism and a response initiated, because the body is dealing with “original parts”, i.e. with active substances with which it is familiar. This enables early intervention in the energy metabolism, optimisation of repair mechanisms, the release of free radicals and many other phenomena.

As defined in claims 14 and 15, the transport system enables a targeted transport and release of the quantities of delivered vitamins, which are needed by the organism for major life functions but which can not be synthesised or can not be synthesised in a sufficient quantity and therefore have to be supplemented through the diet. Apart from their specific functions, vitamins are also elements of coenzymes, which catalyse the cell metabolism.

As defined in claim 16, another advantage is the fact that minerals and trace elements essential to warm-blooded animals can be supplied by means of a particulate transport system. The elements sodium, potassium, magnesium and calcium in physiological concentrations are responsible for maintaining homeostasis. The introduction of synthetic diets in the treatment of metabolic anomalies based on intrinsic genetic defects and the development of intravenous feeding and dialysis treatment of patients with terminal kidney insufficiency are accompanied by iatrogenic risks, which demonstrate the importance of food supplements and if this is not possible the importance of supplementing these elements which occur in the body in minimal concentrations (less than 0.005% of body weight) and the important role they play in the physiology of the human being. However, as more synthetic nutrients are developed, a situation may arise where these elements are absorbed in too excessive quantities. Consequently, food supplements place a systemic strain on the entire organism, giving rise to the occurrence of toxicity, which often does not manifest itself until years later, which more clearly underlines the need for targeted delivery of these elements.

Claim 17 has also proved to be of advantage because a person's general wellbeing can be improved by targeting the transport of these substances. For example, taurine is involved in a whole range of physiological processes, e.g. the conjugation of galenic acid, osmo-regulation, detoxification of xenobiotics, stabilisation of cell membranes, controlling the cellular calcium flow and modulation of neurone excitability. Reduced levels of taurine are associated with degeneration of the retina, retarded growth and cardiomyopathy.

On the basis of the method embodied by claim 18, a deficiency of essential amino acids, which can lead to collapse of the physiological functions of the human body, is prevented. Arginine, for example, increases the lymphocyte count and generally promotes the formation of immune-competent cells. It also increases the cytolytic capacity of macrophages and NK-cells. It also plays an important role in the healing of wounds. Histidine acts an anti-allergic and serves as the precursor of histamine. Isoleucine, leucine and valine are important elements of muscle proteins. Lysin is the main element of collagen, carnitine anti-bodies, hormones and enzymes, supports the healing of wounds and promotes healing of Herpes simplex. Methionine is an antioxidant, detoxifies the liver and is essential for the action of selenium (absorption, transport, bio-availability). Phenylalanine has an anti-depressive effect as well as prolonging the action and increasing the activity of endorphins. Threonine is a lipotropic factor. Tryptophan is important for vitamin B3 synthesis and is a precursor of serotonin and melatonin (sleep rhythm).

As outlined through the embodiment defined in claim 19, it is extremely important for the organism to receive an optimal supply of essential fatty acids, primarily during growth and in the second half of life. This is responsible for the elasticity of the membranes of all body cells, for example, and for the mitochondria, and ensures cell rejuvenation. They occur in the gonads and form the building blocks of the body's natural hormone production in the endocrine gland system but also in the cell tissue. Amongst the fatty acids, an essential role is played by linoleic and α-linolenic acid. They serve as a structural element of the cell membrane and, with the products they produce such as prostaglandin, thromboxane and leukotrienes, control many processes in the organism that are important to life. Arachidonic acid occurs only in animal fats and is a starting product for prostaglandin synthesis. By selectively delivering arachidonic acid to sites at which prostaglandin is formed via the cyclooxygenase-1 metabolic path, a systemic over-supply of arachidonic acid and the negative effects this would cause to rheumatic joint inflammations can be prevented.

The objective of the invention is independently achieved by a method based on the features defined in the characterising part of claim 20. The advantage of this is that a systemic strain on the organism can be avoided because the active substance is released only in the target cell. Systemic side-effects due to too high concentrations of the active substances in cells other than the target cells can be prevented as a result.

The objective of the invention is also independently achieved by using the transport system defined in claim 21. The advantage of this is that a preventive and regenerative effect on the organism can be achieved.

As defined in claim 22, it has advantageously been found that nutrition deficiencies which cause local obstacles to maintaining the physiological equilibrium in the organism can be reduced or eliminated.

As defined din claim 23, it has advantageously been found that the transport system can be delivered directly to the intended site of action by a topical application directly at the intended site of action and the active substance is transported through the cell membrane and not just through the circulation and lymphatic system.

As defined in claim 24, it has proved to be of advantage that the transport system may be used in both pharmaceutical and cosmetic applications as well as in the food and supplement industry.

The invention will be explained in more detail on the basis of examples of embodiments illustrated in the appended drawings.

The drawings show schematic diagrams of:

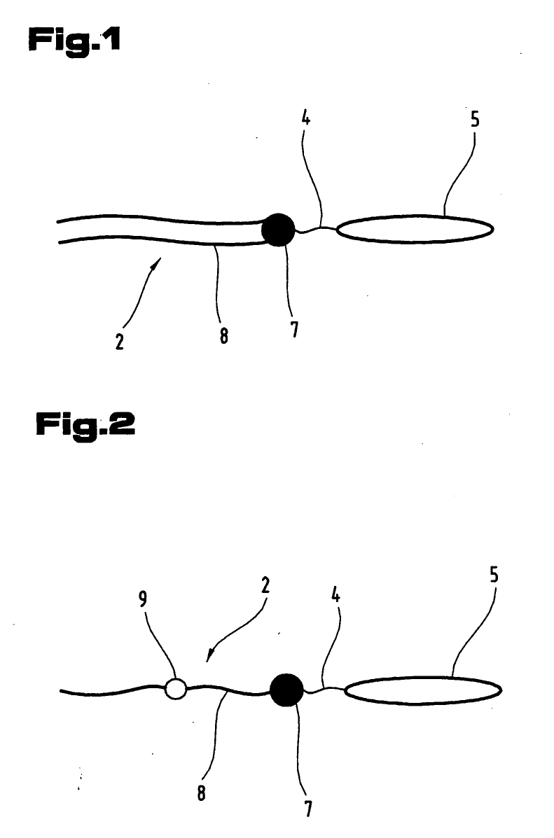

FIG. 1 a hybrid particle;

FIG. 2 a hybrid particle with polymerisable group;

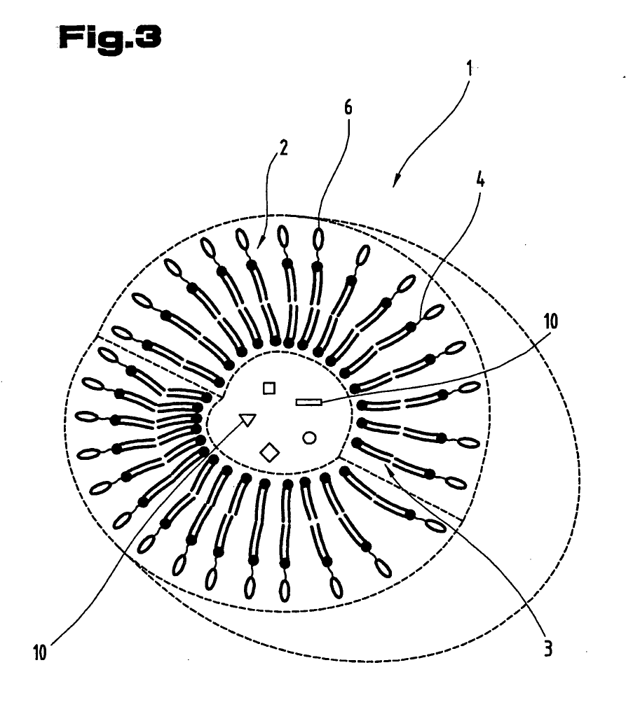

FIG. 3 a transport system.

Firstly, it should be pointed out that the same parts described in the different embodiments are denoted by the same reference numbers and the same component names and the disclosures made throughout the description can be transposed in terms of meaning to same parts bearing the same reference numbers or same component names. Furthermore, the positions chosen for the purposes of the description, such as top, bottom, side, etc, relate to the drawing specifically being described and can be transposed in terms of meaning to a new position when another position is being described.

FIGS. 1 to 3, which will be described together, illustrate a transport system 1 in a biological system.

FIG. 1 illustrates the structure of a bio-active module of transport systems 1. The transport system 1 is made up of hybrid particles 2, consisting of a lipid molecule 3, a spacer unit 4 and a peptide 5. The sequence of the bio-active oligopeptide 6 is coupled by a short spacer unit 4 to the polar head group 7 of the synthetic or natural lipid molecule 3.

FIG. 2 is a schematic diagram of a hybrid particle 2, which has a polymerisable group incorporated in the hydrocarbon chain 8 of the lipid molecule 3.

FIG. 3 illustrates a section through the transport system 1, the contour of the transport system 1 being indicated by a broken line. The transport system 1 consists of a double layer of hybrid particles 2 and lipid molecules 3, where the hybrid particles 2 form the outer layer of the double layer and the lipid molecules 3 form the inner layer. The hybrid particles 2 and lipid molecules 3 organise themselves into a spatial structure and contain substances 10 enclosed in their interior, in particular micro-nutrients, preferably hydrophobic micro-nutrients. In another embodiment, the outer layer of the transport system 1 may also be partially made up of lipid molecules 3, in which case the hybrid particles 2 and the lipid molecules 3 may be arranged in any sequence, for example an oligopeptide 6 is disposed on only every second or third polar head group 7 of a lipid molecule 3. In both embodiments, a polymerisable group 9 may be introduced into the hydrocarbon chain 8 of the lipid molecule 3 itself or the hybrid particles 2.

The active substances 10 used in particular are micro-nutrients, such as provitamins, vitamins, minerals and trace elements, amino acids, fatty acids, polyphenols, hormones and organic extracts and their synthesis products, such as pancreatin, galenic acid, cartilaginous base substances, etc., but naturally also dyes, such as contrasting agents, which have to be transported on a targeted basis for imaging processes in medical investigations.

Hybrid particles 2 of lipid molecules 3, which are modified with specific peptides 5 at the polar head group 7 can be used in a symmetrical layout of the non-polar hydrocarbon chains 8 to build self-organised three-dimensional structures, in particular transport systems 1, such as, for example, liposomes, micelles and oil-in-water emulsions. Important physical-chemical surface properties, such as the surface concentration of the peptide 5, can be exactly controlled.

The oligopeptide sequence is a control sequence for selectively addressing the hybrid particles 2 and/or the transport system 1. Such bio-active peptide sequences, which are bonded on the surface of these systems in a controlled manner, are the basis for an efficient and specifically targeted delivery of the particles to specific cells in specific tissues and a means of preventing non-specific interactions with proteins. Suitable bio-active oligopeptides 6 are ligands, which are typically found in signal proteins of the extra-cellular area.

In particular, the use of transport systems 1 containing hybrid particles 2 with oligopeptides 6 and at least one of the sequences, Gly-Arg-Gly-Asp-Ser-Pro (SEQ ID NO: 1) which form at fibronectin receptor bonding point, Tyr-Ile-Glu-Ser-Arg (SEQ ID NO: 2) which is a laminin receptor bonding site and/or Ala-Asp-Gly-Glu-Ala (SEQ ID NO: 3), which constitutes a collagen receptor bonding site, specifically permit active substances 10 to be selectively transported to skin cells, in particular to fibroblasts.

Suitable means for transporting micro-nutrients selectively to the cells of the eye, in particular to the cells of the retina, are hybrid particles 2 with oligopeptides 6 and at least one of the sequences, Val-Arg-Leu-Leu-Asn-Asn (SEQ ID NO: 4), Val-Arg-Leu Leu-Asn-Asn-Trp-Asp (SEQ ID NO: 5), Gly-Arg-Val-Arg-Leu-Leu-Asn-Asn (SEQ ID NO: 6), which characterise bonding points for the retinol bonding protein at RP 65. Also suitable are hybrid particles 2 with oligopeptides 6 and at least one of the sequences, Met-Thr-Ala-Gly-Ala-Gly (SEQ ID NO: 7), Leu-Ser-Gly-Ala-Leu-Arg (SEQ ID NO: 8), Ile-Val-Ala-Ile-Leu-Ile-Cys-Ile-Leu-Ile-Leu-Leu-Thr-Met-Val-Leu-Leu-Phe-Val-Met-Trp-Met (SEQ ID NO: 9), in which case any section of the amino acid sequence of SEQ ID NO: 9 may be selected as the binding point, e.g. Ile-Val-Ala-Ile-Leu-Ile-Cys-Ile-Leu-Ile-Leu-Leu (SEQ ID NO: 10), Ile-Val-Ala-Ile-Leu-Ile-Cys-Ile-Leu-Ile-Leu-Leu-Thr-Met-Val-Leu-Leu-Phe (SEQ ID NO: 11), Ile-Val-Ala-Ile-Leu-Ile (SEQ ID NO: 12), Cys-Ile-Leu-Ile-Leu-Leu (SEQ ID NO: 13), Thr-Met-Val-Leu-Leu-Phe (SEQ ID NO: 14) and/or Leu-Phe-Val-Met-Trp-Met (SEQ ID NO: 15), which are binding points for R-cadherin for cell-cell adhesion specifically for the retina. In other embodiments several combinations of different sequences of the respective oligopeptides 6 can be used for selective transport to skin cells as well as the eye.

To build functional transport systems 1 comprising self-organising hybrid particles 2 and lipid molecules 3, the strategy followed is one whereby the peptide 5 is firstly bonded to a spacer unit 4, after which the oligopeptide 6 bonded to the spacer unit 4 are jointly coupled with the polar head group 7 of lipid molecules 3. In a second step, the finished ligand-modified hybrid particles 2 are made up into transport system 1. This strategy enables the surface concentration of the oligopeptide 6 to be exactly controlled. By definition, the oligopeptides 6 in this case all sit externally and different emulsion phases can be controllably adjusted on the basis of the hydrodynamic dimensions and physical-chemical surface properties by selecting the concentrations of lipid molecules 3 and ligand-modified lipid molecules 3.

This building concept enables three-dimensional bio-active transport systems 1 to be built, which actively communicate with specific target cells and hence tissue types.

To obtain a spaced arrangement of the oligopeptide 6 on the lipid molecule 3, various substances may be used as a spacer unit 4, e.g. a sequence of amino acids or chemically inert substances such as nano-particles, carbon nano-tubes, nano-threads, colloids, etc., for example.

Apart from being selectively delivered, a controlled release of micro-nutrients from the particles is obtained by incorporating polymerisable groups in the molecular structure of the hybrid particles 2 used and hence due to the cross-linking of the lipid layers in two dimensions. Parameters, such as the size and shape (hydrodynamic dimensions) of the hybrid particles 2, physical-chemical surface properties and the stability of the transport system 1, for example, are each very important for targeting the delivery of micro-nutrients. Polymerisation within the self-organised layers leads to stabilisation and can be used as a means of controlling the release of trapped micro-nutrients. The purpose of controlling the release of micro-nutrients is to ensure that specific micro-nutrients are not only concentrated in the target tissue but also remain constant in a high concentration over a longer period of time.

Such a barrier is built by fixing the lipid molecules 3, which freely diffuse laterally within the mono- or double layers and thus enable a rapid exchange of molecules within and outside the particles. This can be achieved, for example, by incorporating polymerisable groups in the hybrid particles 2, followed by polymerisation or part-polymerisation in two dimensions (within the lipid layers). For example, diacetylene lipids are used, which enable the formation of self-organised structures due to polymerisation, e.g. vesicles that are stable for longer times, stable tubes with very high persistence rates, helical microstructures, etc., whereby diacetylene lipids polymerise under the influence of UV light (λ=254 nm) in an addition reaction. This polymerisation reaction is photochemically controllable and the diacetylene lipids polymerise almost exclusively within a crystalline lipid phase. This results in a polymer which has an extended, conjugated electron system, so that light is absorbed in the visible spectrum. The position of the absorption is dependent on the structure (conformation) of the polymer and can be influenced and shifted by means of various parameters, such as a temperature increase and/or mechanical stress, for example, such as the bonding of a ligand to a corresponding receptor.

Polymerisation results in vesicular transport systems 1 that are stable for a long period, with an absorption maximum at λ=640 nm. Whereas transport systems 1 which do not have any ligands on the surface do not exhibit any significant change in absorption, it may be seen that peptide-modified transport systems 1 take part in a clear interaction with the cells and the absorption maximum shifts significantly. Hydrophobic test substances may also be enclosed in the membrane of the transport system 1 prior to polymerisation and the release of these test substances controlled depending on the quantity of polymerisable lipid molecules 3 in the membrane.

Partially polymerised transport systems 1 can also be produced by adding non-polymerisable hybrid particles 2, which are stable in various physiological environments (pH value, ion intensity, etc.). Lipo-polymers are incorporated in order to obtain steric screening (prevention of non-specific interactions, e.g. with proteins). Fluorescent lipids may also be incorporated to enable better analysis of these structures. The length and concentration of the lipopolymers used specifically influence the bio-activity of the transport systems 1. Optical (light scattering, UV absorption, fluorescence) and microscopic methods (ASM, transmission electron microscopy (TEM), fluorescence) may be used to characterise the morphological and physical-chemical properties and their effects on the parameters, e.g. the efficacy of the quantity of micro-nutrients, stability in various different environments and the controlled release of the targeted transport.

Polymerisable hybrid particles 2 may also be used to obtain thermodynamically stable micro-emulsions in order to produce bio-active transport systems 1 that are as small as possible with a diameter within a lower limit of 5 nm, in particular 10 nm, preferably 15 nm and an upper limit of 180 nm, preferably 160 nm, in particular 140 nm. A range with a lower limit of 20 nm, in particular 30 nm and an upper limit of 120 nm, in particular 100 nm, has also proved to be of advantage. These thermodynamically stable micro-emulsions have as high as possible hydrophobic transport capacity. In addition to the hybrid particles 2, these emulsions may also contain polymerisable lipids and lipo-polymers as well as other hydrophobic components. The phase behaviour of this oil-in-water emulsion is a function of the concentration of the individual temperature parameter and the ion intensity of the medium, the purpose being to produce a thermodynamically stable emulsion phase.

The building principle can also be transferred to “natural” systems, e.g. the structure of peptide-modified phospho-, glyco-, sphingo-lipids, steroids and/or poly-isoprenoids. Accordingly, hybrid particles 2 can be synthesised and characterised on the basis of natural phospholipids. In this case, the phospholipid is directly bonded to the orthogonally protected peptide 5, for example, and the molecule as well as all protective groups are split from the solid phase in a final step. High-pressure liquid chromatography (HPLC) is used for purification purposes. After characterisation by Fourier Transform Infrared Spectroscopy (FTIR), Nuclear Magnetic Resonance spectroscopy (NMR) or Mass Spectroscopy (MS), the resultant modified phospholipids are used in conjunction with non-modified lipids to build bio-active transport systems 1 and emulsions. The same oligopeptides 6 as those characterised above may be used as ligands.

Bio-active planar surfaces, which are produced as a result of the self-organisation of these synthetic hybrid particles 2 followed by modification of solid substrates exhibit a high potential for adhesion. In addition to three-dimensional structures, planar surfaces are also modified with polymerised monolayers of the hybrid particles 2. Cells can become adherent on these surfaces and spread. These surfaces can also be populated with cells again any number of times because they can no longer be dissolved off the surface by polymerisation and cells can therefore be easily removed several times. The results can also be quantified.

Transport systems 1 for active substances 10 may be used to treat illnesses caused by an inadequate supply of micro-nutrients. The transport system 1 may also transport active substances 10 for cosmetic applications and may be applied both orally and topically.

### EMBODIMENT 1

Liposoluble vitamins (tocopherols):

Hybrid particles 2 may be used with oligopeptide sequences such as Gly-Arg-Gly-Asp-Ser-Pro (SEQ ID NO: 1), for example, as a transport system 1 for tocopherols for skin cells, in particular fibroblasts. Targeted delivery of micro-nutrients assists both preventive and regenerative processes, such as maintaining tissue and healing skin wounds. In the case of burns and wound healing, it leads to a reduction in the concentration of antioxidants. This applies in particular to tocopherols which, in terms of quantity, constitute the most important liposoluble vitamins of the skin. Particularly in the case of burns, however, the systemic transport of micro-nutrients is especially poor or even non-existent. By targeting the delivery of tocopherols during regenerative treatment, these nutrients can be directly concentrated in the target cells and released in a controlled manner. The active substances 10 can be retained in different skin layers due to the specific ligands, accumulated and released, thereby producing a local action. The penetration behaviour is also positively influenced because of the small particle size. 3-dimensional skin models may be used as a model for tests.

### EMBODIMENT 2

Carotenoids

For packaging and selectively transporting carotenoids to the cells of the retina, a transport system 1 comprising hybrid particles 2 with the oligopeptide sequence Val-Arg-Leu-Leu-Asn-Asn (SEQ ID NO: 4) is used. Targeted delivery of micro-nutrients assists preventive processes such as avoiding age-related macular degeneration (AMD), for example. In the case of AMD, it has been proven that concentrations of the carotenoids lutein and zeaxanthin in the retina are too low. By packaging carotenoids in particulate systems, provitamins can be selectively transported to the target cells. Cell lines of the retinal epithelia may be used as a modelling system for the eye.

For the sake of good order, it should finally be pointed out that in order to provide a clearer understanding of the transport system 1 for active substances 10, it and its constituent parts are illustrated to a certain extent disproportionately and on an enlarged scale.

The objective underlying the individual inventive solutions may be found in the description.

Above all, the individual embodiments illustrated in FIGS. 1, 2, 3 may be construed as independent solutions proposed by the invention. The objectives and associated solutions proposed by the invention may be found in the detailed descriptions of these drawings.

### LIST OF REFERENCE NUMBERS

1 Transport system
2 Hybrid particles
3 Lipid molecule
4 Spacer unit
5 Peptide
6 Oligopeptide
7 Polar head group
8 Non-polar hydrocarbon chains
9 Polymerisable group
10 Active substances

1-24. (canceled)

25. Transport system for substances containing hybrid particles comprising at least one layer of lipid molecules and at least one ligand, which ligand is a peptide, wherein the peptide has a specific sequence for selective transport purposes and the selectively transported liposome transports at least one micro-nutrient.

26. Transport system according to claim 25, wherein the lipid molecule (3) is bonded to the at least one ligand via a spacer unit (4).

27. Transport system according to claim 26, wherein the spacer unit (4) is made up of amino acids, a chemically inert substance, for example nano-particles such as carbon nano-tubes, nano-threads, colloids, etc.

28. Transport system according to claim 25, wherein the lipid molecules (3) are polymerisable lipids and/or “natural” lipids, for example, such as steroids, glycolipids, phospholipids, sphingolipids, poly-isoprenoids, etc.

29. Transport system according to claim 25, wherein the peptide (5) is an oligopeptide (6).

30. Transport system according to claim 29, wherein the oligopeptide (6) has a length selected from a range with a lower limit of 4 amino acids, preferably 5 amino acids, in particular 6 amino acids, and an upper limit of 18 amino acids, preferably 20 amino acids, in particular 22 amino acids.

31. Transport system according to claim 29, wherein the oligopeptide sequence is complementary to the sequence of a receptor on a cell.

32. Transport system according to claim 29, wherein the oligopeptide (6) has a sequence selected from a group comprising the sequences Gly-Arg-Gly-Asp-Ser-Pro (SEQ ID NO: 1), Tyr-Ile-Glu-Ser-Arg (SEQ ID NO: 2) and/or Ala-Asp-Gly-Glu-Ala (SEQ ID NO: 3).

33. Transport system according to claim 29, wherein the oligopeptide (6) has a sequence, selected from a group comprising the sequences Val-Arg-Leu-Leu-Asn-Asn (SEQ ID NO: 4), Val-Arg-Leu Leu-Asn-Asn-Trp-Asp (SEQ ID NO: 5), Gly-Arg-Val-Arg-Leu-Leu-Asn-Asn (SEQ ID NO: 6), Met-Thr-Ala-Gly-Ala-Gly (SEQ ID NO: 7), Leu-Ser-Gly-Ala-Leu-Arg (SEQ ID NO: 8), Ile-Val-Ala-Ile-Leu-Ile-Cys-Ile-Leu-Ile-Leu-Leu-Thr-Met-Val-Leu-Leu-Phe-Val-Met-Trp-Met (SEQ ID NO: 9), Ile-Val-Ala-Ile-Leu-Ile-Cys-Ile-Leu-Ile-Leu-Leu (SEQ ID NO: 10), Ile-Val-Ala-Ile-Leu-Ile-Cys-Ile-Leu-Ile-Leu-Leu-Thr-Met-Val-Leu-Leu-Phe (SEQ ID NO: 11), Ile-Val-Ala-Ile-Leu-Ile (SEQ ID NO: 12), Cys-Ile-Leu-Ile-Leu-Leu (SEQ ID NO: 13), -Thr-Met-Val-Leu-Leu-Phe (SEQ ID NO: 14) and/or Leu-Phe-Val-Met-Trp-Met (SEQ ID NO: 15).

34. Transport system according to claim 25, wherein the hybrid particles (2) form 3-dimensional structures, such as vesicles, micro-spheres, nano-particles, tubes, etc.

35. Transport system according to claim 25, wherein at least one polymerisable group (9) is incorporated in the hybrid particles (2).

36. Transport system according to claim 25, wherein the at least one micro-nutrient is at least one substance selected from a group comprising provitamins, vitamins, minerals and trace elements, amino acids, fatty acids, polyphenols, hormones and organ extracts or their synthesis products, such as pancreatin, galenic acid, cartilaginous base substance, etc.

37. Transport system according to claim 25, wherein the vitamin is selected from a group comprising natural and synthetic compounds with a retinoid structure (vitamin A), vitamin B complex, ascorbic acids (vitamin C), calciferols (vitamin D), tocopherols (vitamin E), vitamin K, flavonoids and biotin.

38. Transport system according to claim 25, wherein the at least one vitamin is selected from a group comprising retinol, retinyl acetate, retinyl palmitate, 3,4-didehydroretinol (vitamin A2), retinal, retinic acid and provitamins, such as α-, β-, γ-carotin, lutein, zeaxanthin, thiamin (vitamin B1) or thiamin hydrochloride or thiamin monomitrate, riboflavin (vitamin B2) or sodium-riboflavin-5-phosphate, niacin (vitamin B3) or nicotinic acid or neacin, pantothenic acid (Vitamin B5) or. calcium-D-pantothenate or sodium-D-pantothenate or D-panthenol, pyridoxin (vitamin B6) or pyridoxin hydrochloride or. pyridoxin-5-phosphate or pyridoxin dipalmitate or pyridoxal phosphate, folic acid (vitamin B9) or pteroyl glutamic acid, cobalamin (vitamin B12) or cyano-cobalamin or hydroxycobalamin, biotin, choline, inosit and p-aminobenzoic acid, L-ascorbic acid, sodium-L-ascorbate, calcium-L-ascorbate, potassium-L-ascorbate and L-ascorbyl-6-Palmitate, ergocalciferol (vitamin D2), cholecalciferol (vitamin D3), 1,25-dihydroxycholecalciferol and the provitamins ergosterol or 7-dehydrocholesterol, D-α-tocopherol, DL-α-tocopherol, D-α-tocopheryl acetate, DL-α-tocopheryl acetate and D-α-tocopheryl acid succinate, phylloquinone (vitamin K1) , menoquinone (vitamin K2), menadion (vitamin K3) and menadione hydroxiquinone (vitamin K4).

39. Transport system according to claim 25, wherein the at least one mineral and the at least one trace element are selected, according to their importance for the organism, from a group comprising Na, K, Mg, Ca, Fe, I, Cu, Mn, Zn, Co, Mo, Se, Cr, F, Si, Ni, As, Sn, V, P, Cl, B, Al and Br.

40. Transport system according to claim 25, wherein the at least one component is selected from a group comprising coenzyme Q-10, quercetin, bromelain, inositol, choline, pycnogenol, carnitine, taurine, mesoinosit.

41. Transport system according to claim 25, wherein the at least one essential amino acid is selected from a group comprising histidine, isoleucine, leucine, lysin, methionine, phenyl alanine, threonine, tryptophan, valine and arginine.

42. Transport system according to claim 25, wherein the at least one fatty acid is selected from a group comprising linoleic acid, linolenic acid and arachidonic acid.

43. Method of transporting active substances, wherein the transport system according to claim 25 is used.

44. Transport system according to claim 25 for use as a medicament.

45. Use of the transport system according to claim 44 for producing a medicament for the treatment of nutritional deficiencies.

46. Use of the transport system according to claim 45 for topical and oral application.

47. Use of the transport system according to claim 25, wherein it is used in the pharmaceutical and food industry.


SEQUENCE LISTING (15 sequences)

SEQ ID NO: 1 - protein, 6 aa, Artificial sequence
  Chemically Synthesized
GRGDSP

SEQ ID NO: 2 - protein, 5 aa, Artificial sequence
  Chemically Synthesized
YIESR

SEQ ID NO: 3 - protein, 5 aa, Artificial sequence
  Chemically Synthesized
ADGEA

SEQ ID NO: 4 - protein, 6 aa, Artificial sequence
  Chemically Synthesized
VRLLNN

SEQ ID NO: 5 - protein, 8 aa, Artificial sequence
  Chemically Synthesized
VRLLNNWD

SEQ ID NO: 6 - protein, 8 aa, Artificial sequence
  Chemically Synthesized
GRVRLLNN

SEQ ID NO: 7 - protein, 6 aa, Artificial sequence
  Chemically Synthesized
MTAGAG

SEQ ID NO: 8 - protein, 6 aa, Artificial sequence
  Chemically Synthesized
LSGALR

SEQ ID NO: 9 - protein, 22 aa, Artificial sequence
  Chemically Synthesized
IVAILICILILLTMVLLFVMWM

SEQ ID NO: 10 - protein, 12 aa, Artificial sequence
  Chemically Synthesized
IVAILICILILL

SEQ ID NO: 11 - protein, 18 aa, Artificial sequence
  Chemically Synthesized
IVAILICILILLTMVLLF

SEQ ID NO: 12 - protein, 6 aa, Artificial sequence
  Chemically Synthesized
IVAILI

SEQ ID NO: 13 - protein, 6 aa, Artificial sequence
  Chemically Synthesized
CILILL

SEQ ID NO: 14 - protein, 6 aa, Artificial sequence
  Chemically Synthesized
TMVLLF

SEQ ID NO: 15 - protein, 6 aa, Artificial sequence
  Chemically Synthesized
LFVMWM